(19) United States
(12) Patent Application Publication
(10) Pub. No.: US 2020/0179524 A1
(43) Pub. Date: Jun. 11, 2020

(54) METHODS OF TREATING MULTIPLE MYELOMA CANCERS EXPRESSING HIGH LEVELS OF EPO-RECEPTOR USING PSA-EPO

(71) Applicant: Lipoxen Technologies Limited, London (GB)

(72) Inventor: Dmitry Genkin, St. Petersburg (RU)

(73) Assignee: Lipoxen Technologies Limited, London (GB)

(21) Appl. No.: 16/608,781

(22) PCT Filed: Apr. 25, 2018
(86) PCT No.: PCT/EP2018/060562

§ 371 Date: Oct. 25, 2019

Related U.S. Application Data
(60) Provisional application No. 62/489,914, filed on Apr. 25, 2017.

(51) Int. Cl. A61K 47/60; A61K 38/18; A61K 47/61; A61P 35/00

(52) U.S. Cl. CPC A61K 47/60; A61P 35/00; A61K 47/61; A61K 38/1816

(57) ABSTRACT

The present invention demonstrates that erythropoietin (EPO)-receptor (EPOR) is a malignant myeloma biomarker of sensitivity to EPO treatment and, itself a target for myeloma treatment. A low EPOR level in a myeloma cells of the subject indicates non-response to EPO treatment. Patients having high EPOR level in myeloma cells can be effectively treated with EPO, in particular an EPO derivatized with polysialic acid.

### RELATED APPLICATIONS

This application claims priority and is entitled to the filing date of U.S. provisional application Ser. No. 62/489,914 filed on Apr. 25, 2017. The contents of the aforementioned application(s) are incorporated herein by reference.

### BACKGROUND

Multiple myeloma is a hematological cancer caused by the presence of malignant plasma cells in bone marrow. The patients are characterized by symptoms of osteoporotic or osteolytic bone disease, kidney dysfunction, recurrent infections and anemia. Multiple myeloma is considered treatable but incurable. Remissions may be brought about with steroids, chemotherapy, thalidomide or lenalidomide, and stem cell transplant. Bisphosphonates and radiation therapy are sometimes used to reduce pain from bone lesions.

Globally, multiple myeloma affected about 427,000 people in 2015 and resulted in about 79,000 deaths. In the United States it develops in 6.5 per 100,000 people per year and 0.7% of people are affected at some point in their life. It usually occurs around the age of 61 and is more common in men than women. Without treatment, typical survival is seven months. With current treatments, survival is usually 4-5 years. This gives a five-year survival rate of about 49%.

Erythropoietin is an essential hormone for red blood cell production. Without it, definitive erythropoiesis does not take place. Under hypoxic conditions, the kidney will produce and secrete erythropoietin to increase the production of red blood cells by targeting CFU-E, proerythroblast and basophilic erythroblast subsets in the differentiation. Erythropoietin has its primary effect on red blood cell progenitors and precursors (which are found in the bone marrow in humans) by promoting their survival through protecting these cells from apoptosis. EPO alpha, beta and gamma all have the same 165 amino acid sequence, but differ in their glycosylation pattern.

Erythropoietins available for use as therapeutic agents are produced by recombinant DNA technology in cell culture, and include EPOGEN/PROCRIT® (epoetin alpha) and ARANESP® (darbepoetin alpha); they are used in treating anemia resulting from chronic kidney disease, chemotherapy induced anemia in patients with cancer, inflammatory bowel disease (Crohn's disease and ulcerative colitis) and myelodysplasia from the treatment of cancer (chemotherapy and radiation). The package inserts include boxed warnings of increased risk of death, myocardial infarction, stroke, venous thromboembolism, and tumor recurrence.

It has recently been reported that a substantial fraction (40-50%) of myeloma cell clones may express the EPO-receptor (EPOR). Furthermore, myeloma cell lines expressing EPOR are sensitive to apoptosis when treated with rhuEPO (Våtsween et al., 2016, J Hematol Oncol. 9(1):75). Myeloma patients are generally anemic and may today be unsystematically treated with EPO to improve erythropoiesis. However, as described above, EPO can have toxic side effects and is not uniformly prescribed for anemia in multiple myeloma patients, nor is it prescribed as a primary treatment for multiple myeloma itself

In contrast, the present invention demonstrates that EPO treatment also has direct anti-myeloma activity in primary cells, but only in the sub-set of patients that are characterized by high EPOR expression on myeloma cells. For this sub-population of multiple myeloma patients, treatment of the primary cancer with EPO or derivatives with EPO activity is now provided, optionally in combination with a companion diagnostic to assess the level of EPOR expression.

Epoetin alpha and beta exhibit some differences in their pharmacokinetics, possibly due to differences in glycosylation and in the formulation of the commercial preparations. Epoetin alpha is slowly and incompletely absorbed following subcutaneous injection and a bioavailability of about 10 to 50% relative to intravenous administration has been reported. Epoetin beta is also slowly and incompletely absorbed and its absolute bioavailability has been reported to be around 40%.

Attempts have been made to derivatise EPO to improve its pharmacokinetic properties. There is a product under development by Roche, known as CERA (Constant Erythropoiesis Receptor Activator), which is a polyethylene glycol derivatised form of EPO. This has been shown to have a longer half-life than EPO, reducing the necessity of frequent injections. A further novel erythropoiesis stimulating agent is Hematide, a novel, PEGylated, synthetic peptide for the treatment of anaemia associated with chronic kidney disease and cancer. This is described further by Fan et al (2006).

Other forms of EPO have also been developed, such as darbepoetin, a hyperglycosylated analogue of recombinant human erythropoietin which has around a three-fold longer terminal half-life after i.v. administration than recombinant human EPO and the native hormone.

EP 1219636 describes modified muteins of EPO produced from a microorganism with a prolonged plasma half-life in the circulation. A cell-free protein synthesis technique is used to produce a mutein of EPO with an unnatural amino acid which may be reacted with a modifier such as PEG or a polysaccharide. Generally, PEG is attached to a free sulfhydryl group in the muteins of EPO. U.S. Pat. No. 7,128,913 is directed to N-terminal conjugates of EPO with PEG. The conjugates have an increased circulating half-life and plasma residence time.

U.S. Pat. No. 7,074,755 also addresses the problem of providing improved biologically active EPO conjugate compositions. The EPO is covalently conjugated to a non-antigenic hydrophilic polymer covalently linked to an organic molecule that increases the circulating serum half-life of the composition. The water-soluble polymer may be a polyalkylene oxide, a polyamide, or a carbohydrate, amongst others.

However, there has been no published work to date describing the derivatisation of EPO with anionic polysaccharides such as polysialic acid (PSA).

Polysialic acids (PSAs) are naturally occurring unbranched polymers of sialic acid produced by certain bacterial strains and in mammals in certain cells. They can be produced in various degrees of polymerisation from n=about 80 or more sialic acid residues down to n=2 by limited acid hydrolysis or by digestion with neuraminidases, or by fractionation of the natural, bacterially derived forms of the polymer.

In recent years, the biological properties of polysialic acids, particularly those of the alpha-2,8 linked homopolymeric polysialic acid, have been exploited to modify the pharmacokinetic properties of protein and low molecular weight drug molecules. Polysialic acid derivatisation gives rise to dramatic improvements in circulating half-life for a number of therapeutic proteins including catalase and asparaginase, and also allows such proteins to be used in the face of pre-existing antibodies raised as an undesirable (and sometimes inevitable) consequence of prior exposure to the therapeutic protein. The alpha-2,8 linked polysialic acid offers an attractive alternative to PEG, being an immunologically invisible biodegradable polymer which is naturally part of the human body, and which degrades, via tissue neuraminidases, to sialic acid, a non-toxic saccharide (see, e.g., PCT/GB2007/002841 hereby incorporated by reference in its entirety).

The present invention thus provides water soluble polymer conjugates of EPO for the treatment of EPOR-expressing multiple myelomas. The derivatives are useful for improving the stability, pharmacokinetics and pharmacodynamics of EPO.

### SUMMARY OF THE INVENTION

In one aspect, the present invention provides method of treating multiple myeloma in a subject in need thereof, the method comprising administering to the subject a pharmaceutical composition comprising an effective amount of an EPO conjugated with a water soluble polymer, wherein a myeloma sample from the subject has erythropoietin receptor expressed on the cell surface and wherein the EPOR receptor expression is higher than a predetermined threshold level.

In one embodiment, the erythropoietin receptor expression in the myeloma sample is measured using an immunoassay. In one embodiment, the predetermined threshold level by ELISA is at least or above 0.5 pg/mg of sample protein, 1.5 pg/mg of sample protein, 5 pg/mg of sample protein, 50 pg/mg of sample protein or higher. In one embodiment, the predetermined threshold level is a EpoR receptor density per cell and is at least 100 EpoR receptor copies per cell. In one embodiment, the predetermined threshold level is a percent of EpoR positive myeloma cells (ICH) in the sample at least over 5%, 10% to 25% or greater than 25%. In one embodiment, the erythropoietin receptor gene product expression in the myeloma sample is measured using PCR.

In one embodiment, the EPO is conjugated to the water soluble polymer via a linking group. In one embodiment, water soluble polymer is PEG, poly(2-ethyl 2-oxazoline), poly[oligo(ethylene glycol) methyl methacrylate], polyoxazoline, poly(N-(2-hydroxypropyl) methacrylamide, polyglycerol, poly(N-vinylpyrrolidone), polycarbonate, poly(carboxybetaine methacrylate), poly(sulfobetaine methacrylate) or poly(2-methyacryloyloxyethyl phosphorylcholine). In one embodiment, the EPO is linked via an amine group at the N-terminus to a polysaccharide. In one embodiment, the polysaccharide is selected from the group consisting of polysialic acid, heparin, hyaluronic acid, dextran, dextrin, hydroxyethyl starch, and chondroitin sulphate. In one embodiment, the polysaccharide is polysialic acid. In one embodiment, the polysialic acid is attached to the N-terminus of EPO at the reducing terminal unit of the polysialic acid.

In one embodiment, the EPO has at least 95% sequence identity to an amino acid sequence comprising SEQ ID NO:1. In one embodiment, the EPO has an amino acid sequence comprising SEQ ID NO: 1. In one embodiment, the EPO has an amino acid sequence comprising residues 28-193 of SEQ ID NO: 1.

In one embodiment, the subject has not received EPO treatment for anemia. In one embodiment, the patient is suffering from concurrent anemia.

In one embodiment, the EPO conjugate has a systemic clearance at least 50% or lower as compared to EPO that is not conjugated with a water soluble polymer

In one aspect, the invention provides a companion therapeutic-diagnostic kit comprising an antibody or EPOR binding partner for detecting erythropoietin receptor expression in a myeloma sample and a pharmaceutical composition comprising EPO conjugated to a polysialic acid.

In one aspect, the invention provides a companion therapeutic-diagnostic kit comprising nucleic acid primers for detecting erythropoietin receptor gene product expression in a myeloma sample and a pharmaceutical composition comprising EPO conjugated to a polysialic acid.

### DESCRIPTION OF THE DRAWINGS

Not applicable.

### DETAILED DESCRIPTION OF THE INVENTION

The present invention provides that the hypoxia-dependent erythropoietin (EPO)-receptor (EPOR) is a multiple myeloma biomarker of sensitivity to EPO treatment and, itself a target for myeloma treatment. A low EPOR expression level in a myeloma cells of the subject indicates non-response to EPO treatment. Patients having high EPOR expression level in myeloma cells can be effectively treated with EPO, in particular an EPO conjugated to a water soluble polymer such as a polysialic acid.

Treatment of EPOR-expressing multiple myeloma with an EPO-water soluble polymer conjugate provides several advantages. First, the EPO-water soluble polymer conjugate is expected to have clearance in a subject's body at least 50%, 45%, 40%, 35%, 30%, 25%, 20%, 15%, or 10% or lower compared to DNase that is not conjugated with a water soluble polymer. Therefore, the EPO conjugate is more effective because when the cancer cell is triggered to die the EPO-conjugate can go to the next cell. In contrast, non-conjugated EPO is degraded. Clearance is a pharmacokinetic measurement of the volume of plasma from which a substance is completely removed per unit time; the usual units are mL/min. The quantity reflects the rate of drug elimination divided by plasma concentration.

Another advantage is that less EPO-conjugate is needed than EPO alone. As described above, EPO is known to have significant side effects in the treatment of anemia. However, since the EPO-conjugate has improved pharmacokinetic attributes, including slower clearance, it can be administered at a lower dose, thus resulting in a reduction in adverse events.

The level of expression or overexpression of EPOR in a patient with multiple myeloma can be determined using either protein or nucleic acid detection assays on a myeloma sample, typically a bone marrow biopsy sample. The level of EPOR expression in the patient sample is compared to the level of EPOR expression in a reference non-EPO responsive multiple myeloma sample or low-EPOR expressing multiple myeloma sample. If the level of EPOR expression is at least about 10%, 15%, 20%, 25%, 30%, 35%, 40%, 45%, 50%, 55%, 60% 65%, 70%, 75%, 80%, 85%, 90%, 95%, 100%, 200%, 300%, 400% 500% or higher than that of the reference sample, the patient is a candidate for EPO treatment of the primary cancer.

### EPO Compositions

Hereinafter, when using the term EPO, it also encompasses EPO-like proteins. By EPO-like protein is meant a protein which has an activity equivalent to that of EPO. EPO regulates erythrocyte production, as detailed above. The activity of EPO or an EPO-like protein can be measured using a standard assay as described in Krystal (1983). The activity of EPO samples in inducing proliferation in vitro of erythrocyte progenitor cells isolated from the spleen of a mouse is measured. The mice have previously been rendered anaemic artificially through I.P. injection of phenylhydrazine. In the assay, EPO is added to erythrocyte progenitors and the rate of DNA replication is measured by determining the rate of incorporation of ³H-thymidine. A protein is classified as “EPO-like” if it induces 10-200% of the rate of replication compared to standard EPO from NIBSC. Typically, an EPO-like protein has at least 35% of the activity of standard EPO, and preferably, at least 50% of the activity of standard EPO.

Mutants of EPO which have the requisite activity, as detailed above, may also be used. An “EPO-like” protein may also be referred to as an “EPO-homologue”. Whether two sequences are homologous is routinely calculated using a percentage similarity or identity, tents that are well known in the art. Sequences should be compared to SEQ ID NO:1, which is human EPO precursor with swissprot accession number PO1588. The active EPO is residues 28-193 of this sequence. EPO homologue sequences may either be compared to the whole of SEQ ID NO:1, or residues 28-193 thereof. Preferably, EPO homologue sequences are compared to the active EPO, i.e., residues 28-193.

```
SEQ ID NO: 1
Met Gly Val His Glu Cys Pro Ala Trp Leu Trp Leu
1 5 10
Leu Leu Ser Leu Leu Ser Leu Pro Leu Gly Leu Pro
                15 20
Val Leu Gly Ala Pro Pro Arg Leu Ile Cys Asp Ser
25 30 35
Arg Val Leu Glu Arg Tyr Leu Leu Glu Ala Lys Glu
                        40 45
Ala Glu Asn Ile Thr Thr Gly Cys Ala Glu His Cys
        50 55 60
Ser Leu Asn Glu Asn Ile Thr Val Pro Asp Thr Lys
                                65 70
Val Asn Phe Tyr Ala Trp Lys Arg Met Glu Val Gly
                75 80
Gln Gln Ala Val Glu Val Trp Gln Gly Leu Ala Leu
85 90 95
Leu Ser Glu Ala Val Leu Arg Gly Gln Ala Leu Leu
                        100 105
Val Asn Ser Ser Gln Pro Trp Glu Pro Leu Gln Leu
        110 115 120
His Val Asp Lys Ala Val Ser Gly Leu Arg Ser Leu
                                125 130
Thr Thr Leu Leu Arg Ala Leu Gly Ala Gln Lys Glu
                135 140
Ala Ile Ser Pro Pro Asp Ala Ala Ser Ala Ala Pro
145 150 155
Leu Arg Thr Ile Thr Ala Asp Thr Phe Arg Lys Leu
                        160 165
Phe Arg Val Tyr Ser Asn Phe Leu Arg Gly Lys Leu
        170 175 180
Lys Leu Tyr Thr Gly Glu Ala Cys Arg Thr Gly Asp
                                185 190
Arg
```

In this invention, homologues have 50% or greater similarity or identity at the nucleic acid or amino acid level, preferably 60%, 70%, 80% or greater, more preferably 90% or greater, such as 95% or 99% identity or similarity at the amino acid level. A number of programs are available to calculate similarity or identity; preferred programs are the BLASTn, BLASTp and BLASTx programs, run with default parameters (available on the NCBI-NIH database). For example, 2 amino acid sequences may be compared using the BLASTn program with default parameters (score=100, word length=11, expectation value=11, low complexity filtering=on). The above levels of homology may be calculated using these default parameters.

### EPO Derivatized with Polysaccharide

As described above, in addition to the naturally occurring or engineered glycosylation pattern of EPO produced by cells expressing EPO, the EPO molecule may be chemically derivatized with a polysaccharide. Preferably, the polysaccharide has at least 2, more preferably at least 5, most preferably at least 10, for instance at least 50 or more saccharide units.

The term “water-soluble” refers to moieties that have some detectable degree of solubility in water. Methods to detect and/or quantify water solubility are well known in the art. Exemplary water-soluble polymers include peptides, saccharides, poly(ethers), poly(amines), poly(carboxylic acids) and the like. Peptides can have mixed sequences of be composed of a single amino acid, e.g., poly(lysine). An exemplary polysaccharide is poly(sialic acid). An exemplary poly(ether) is poly(ethylene glycol). Poly(ethylene imine) is an exemplary polyamine, and poly(acrylic) acid is a representative poly(carboxylic acid). The water soluble polymer can be PEG, poly(2-ethyl 2-oxazoline), poly[oligo(ethylene glycol) methyl methacrylate], polyoxazoline, poly(N-(2-hydroxypropyl)) methacrylamide, polyglycerol, poly(N-vinylpyrrolidone), polycarbonate, poly(carboxybetaine methacrylate), poly(sulfobetaine methacrylate) or poly(2methyacryloyloxyethyl). phosphorylcholine). The polysaccharide is selected from polysialic acid, dextran, dextrin, heparin, hyaluronic acid, hydroxyethyl starch and chondroitin sulphate. Preferably, the polysaccharide is polysialic acid and consists substantially only of sialic acid units. However, the polysaccharide may have units other than sialic acid in the molecule. For instance, sialic acid units may alternate with other saccharide units. Preferably, however, the polysaccharide consists only of units of sialic acid. The polymer backbone can be linear or branched. Branched polymer backbones are generally known in the art. Typically, a branched polymer has a central branch core moiety and a plurality of linear polymer chains linked to the central branch core.

Preferably, the derivatized compound is an N-terminal derivative of EPO or of an EPO-like protein, that is, the polysaccharide is associated with the EPO at its N-terminus. Alternatively, however, the polysaccharide may be associated with the EPO or EPO-like protein at a mid-chain amino acid, such as at the side chain of a lysine, cysteine, aspartic acid, arginine, glutamine, tyrosine, glutamic acid or histidine. Typically, the side chain is of a lysine of cysteine amino acid.

Preferably the polysaccharide has a terminal sialic acid group, and as detailed above, is more preferably a polysialic acid, that is a polysaccharide comprising at least 2 sialic acid units joined to one another through α-2-8 or α-2-9 linkages. A suitable polysialic acid has a weight average molecular weight in the range 2 to 50 kDa, preferably in the range 5 to 50 kDa. Most preferably, the polysialic acid is derived from a bacterial source, for instance polysaccharide B of E. coli KI, Maraxella liquefaciens or Pasteurella aeruginosa or K92 polysaccharide from E. coli K92 strain. It is most preferably colominic acid from E. coli K1.

The polysialic acid may be in the form of a salt or the free acid. It may be in a hydrolysed form, such that the molecular weight has been reduced following recovery from a bacterial source.

The polysaccharide, which is preferably polysialic acid may be material having a wide spread of molecular weights such as having a polydispersity of more than 1.3, for instance as much as 2 or more. Preferably the polydispersity (p.d.) of molecular weight is less than 1.3, more preferably less than 1.2, for instance less than 1.1. The p.d. may be as low as 1.01.

The EPO may be derivatised with more than one anionic polysaccharide. For instance, the EPO may be derivatised at both its N-terminus and at an internal amino acid side chain. The side chains of lysine, cysteine, aspartic acid, arginine, glutamine, tyrosine, glutamic acid, serine and histidine, for instance, may be derivatised by an anionic polysaccharide. The EPO may also be derivatised on a glycon unit. However, in a preferred embodiment of this invention, the EPO is derivatised at its N-terminus only.

The derivatized compound may be a covalently-linked conjugate between the EPO and an anionic polysaccharide. The EPO may be covalently linked to the polysaccharide at its N-terminal amino acid. The covalent linkage may be an amide linkage between a carboxyl group and an amine group. Another linkage by which the EPO could be covalently bonded to the polysaccharide is via a Schiff base. Suitable groups for conjugating to amines are described further in WO2006/016168. The DNase can be conjugated to the polysaccharide via a reactive aldehyde on the polysaccharide. Chemistry suitable for preparing a polysaccharide with a reactive aldehyde at the reducing terminal of a polysaccharide is described in WO 05/016974. The process involves a preliminary selective oxidation step followed by reduction and then further oxidation to produce a compound with an aldehyde at the reducing terminal and a passivated non-reducing end.

Suitable linkers are derived from N-maleimide, vinylsulphone, N-iodoacetamide, orthopyridyl or N-hydroxysuccinimide-containing reagents. The linker may also be biostable or biodegradable and comprise, for instance, a polypeptide or a synthetic oligomer. The linker may be derived from a bifunctional moiety, as further described in WO2005/016973. A suitable bifunctional reagent is, for instance, Bis-NHS.

### Determination of EPOR Overexpression

Overexpression” refers to RNA or protein expression of EPOR in a tissue that is significantly higher that RNA or protein expression of in a control or reference tissue sample. In one embodiment, the tissue sample is autologous. Cancerous test tissue samples associated with EPO treatment responsiveness typically have at least two-fold higher expression of EPOR mRNA or protein, often up to three, four, five, eight, ten or more fold higher expression of EPOR protein in comparison to cancer tissues from patients who are not EPO responsive or to normal (i.e., non-cancer) tissue samples. Such differences may be readily apparent when viewing the bands of gels with approximately similarly loaded with test and controls samples. The terms “overexpress,” “overexpression” or “overexpressed” interchangeably refer to a gene that is transcribed or translated at a detectably greater level, usually in a cancer cell, in comparison to a normal or cancerous cell of the same type. Overexpression therefore refers to both overexpression of protein and RNA (due to increased transcription, post transcriptional processing, translation, post translational processing, altered stability, and altered protein degradation), as well as local overexpression due to altered protein traffic patterns (increased nuclear localization), and augmented functional activity, e.g., as in an increased enzyme hydrolysis of substrate. Overexpression can also be by 50%, 60%, 70%, 80%, 90% or more (2-fold, 3-fold, 4-fold) in comparison to a non-cancerous cell or cancerous of the same type. The overexpression may be based upon visually detectable or quantifiable differences observed using immunohistochemical or nucleic acid based methods to detect EPOR protein or nucleic acid.

In one embodiment, overexpression is determined using a predetermined threshold level of EPOR expression. For example, in on e embodiment, the predetermined threshold level by ELISA is at least or above 0.5pg/mg of sample protein, 1.5 pg/mg of sample protein, 5 pg/mg of sample protein, 50 pg/mg of sample protein or higher. In another embodiment, the predetermined threshold level is a EpoR receptor density per cell and is at least 100 EpoR receptor copies per cell. In another embodiment, the predetermined threshold level is a percent of EpoR positive myeloma cells (ICH) in the sample at least over 5%, 10% to 25% or greater than 25%.

In some embodiments, the invention provides a companion diagnostic kit comprising: an agent which detects the level of EPOR; method or instructions for using the agent for detecting the level of EPOR in a sample; a control or reference standard; and instructions to provide guidance for carrying out an assay embodied by the kit and for making a determination of the level of EPOR based upon that assay.

### Pharmaceutical Compositions and Administration

The present specification also provides a pharmaceutical composition for the administration to a subject. The pharmaceutical composition disclosed herein may further include a pharmaceutically acceptable carrier, excipient, or diluent. As used herein, the term “pharmaceutically acceptable” means that the composition is sufficient to achieve the therapeutic effects without deleterious side effects, and may be readily determined depending on the type of the diseases, the patient's age, body weight, health conditions, gender, and drug sensitivity, administration route, administration mode, administration frequency, duration of treatment, drugs used in combination or coincident with the composition disclosed herein, and other factors known in medicine.

The pharmaceutical composition including the EPO molecule disclosed herein may further include a pharmaceutically acceptable carrier. For oral administration, the carrier may include, but is not limited to, a binder, a lubricant, a disintegrant, an excipient, a solubilizer, a dispersing agent, a stabilizer, a suspending agent, a colorant, and a flavorant. For injectable preparations, the carrier may include a buffering agent, a preserving agent, an analgesic, a solubilizer, an isotonic agent, and a stabilizer. For preparations for topical administration, the carrier may include a base, an excipient, a lubricant, and a preserving agent.

The disclosed compositions may be formulated into a variety of dosage forms in combination with the aforementioned pharmaceutically acceptable carriers. For example, for oral administration, the pharmaceutical composition may be formulated into tablets, troches, capsules, elixirs, suspensions, syrups or wafers. For injectable preparations, the pharmaceutical composition may be formulated into an ampule as a single dosage form or a multidose container. The pharmaceutical composition may also be formulated into solutions, suspensions, tablets, pills, capsules and long-acting preparations.

On the other hand, examples of the carrier, the excipient, and the diluent suitable for the pharmaceutical formulations include, without limitation, lactose, dextrose, sucrose, sorbitol, mannitol, xylitol, erythritol, maltitol, starch, acacia rubber, alginate, gelatin, calcium phosphate, calcium silicate, cellulose, methylcellulose, microcrystalline cellulose, polyvinylpyrrolidone, water, methylhydroxybenzoate, propylhydroxybenzoate, talc, magnesium stearate and mineral oils. In addition, the pharmaceutical formulations may further include fillers, anti-coagulating agents, lubricants, humectants, flavorants, and antiseptics.

The pharmaceutical composition disclosed herein may have any formulation selected from the group consisting of tablets, pills, powders, granules, capsules, suspensions, liquids for internal use, emulsions, syrups, sterile aqueous solutions, non-aqueous solvents, lyophilized formulations and suppositories.

Further, the composition may be formulated into a single dosage form suitable for the patient's body, and preferably is formulated into a preparation useful for protein drugs according to the typical method in the pharmaceutical field so as to be administered by an oral or parenteral route such as through skin, intravenous, intramuscular, intra-arterial, intramedullary, intramedullary, intraventricular, pulmonary, transdermal, subcutaneous, intraperitoneal, intranasal, intracolonic, topical, sublingual, vaginal, or rectal administration, but is not limited thereto.

The composition may be used by blending with a variety of pharmaceutically acceptable carriers such as physiological saline or organic solvents. In order to increase the stability or absorptivity, carbohydrates such as glucose, sucrose or dextrans, antioxidants such as ascorbic acid or glutathione, chelating agents, low molecular weight proteins or other stabilizers may be used.

The pharmaceutical composition disclosed herein is expected to have longer in-vivo duration of efficacy and titer, thereby remarkably reducing the number and frequency of administration thereof.

Moreover, the pharmaceutical composition may be administered alone or in combination or coincident with other pharmaceutical formulations showing prophylactic or therapeutic efficacy.

The therapeutic method of the present specification may include the step of administering the composition including the EPO protein at a pharmaceutically effective amount. The total daily dose should be determined through appropriate medical judgment by a physician, and administered once or several times. The specific therapeutically effective dose level for any particular patient may vary depending on various factors well known in the medical art, including the kind and degree of the response to be achieved, concrete compositions according to whether other agents are used therewith or not, the patient's age, body weight, health condition, gender, and diet, the time and route of administration, the secretion rate of the composition, the time period of therapy, other drugs used in combination or coincident with the composition disclosed herein, and like factors well known in the medical arts.

In one embodiment, the dose of the composition may be administered daily, semi-weekly, weekly, bi-weekly, or monthly. The period of treatment may be for a week, two weeks, a month, two months, four months, six months, eight months, a year, or longer. The initial dose may be larger than a sustaining dose. In one embodiment, the dose ranges from a weekly dose of at least 0.01 mg, at least 0.25 mg, at least 0.3 mg, at least 0.5 mg, at least 0.75 mg, at least 1 mg, at least 1.25 mg, at least 1.5 mg, at least 2 mg, at least 2.5 mg, at least 3 mg, at least 4 mg, at least 5 mg, at least 6 mg, at least 7 mg, at least 8 mg, at least 9 mg, at least 10 mg, at least 15 mg, at least 20 mg, at least 25 mg, at least 30 mg, at least 35 mg, at least 40 mg, at least 50 mg, at least 55 mg, at least 60 mg, at least 65 mg, or at least 70 mg. In one embodiment, a weekly dose may be at most 0.5 mg, at most 0.75 mg, at most 1 mg, at most 1.25 mg, at most 1.5 mg, at most 2 mg, at most 2.5 mg, at most 3 mg, at most 4 mg, at most 5 mg, at most 6 mg, at most 7 mg, at most 8 mg, at most 9 mg, at most 10 mg, at most 15 mg, at most 20 mg, at most 25 mg, at most 30 mg, at most 35 mg, at most 40 mg, at most 50 mg, at most 55 mg, at most 60 mg, at most 65 mg, or at most 70 mg. In a particular aspect, the weekly dose may range from 0.25 mg to 2.0 mg, from 0.5 mg to 1.75 mg. In an alternative aspect, the weekly dose may range from 10 mg to 70 mg.

### Definitions

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Methods for obtaining (e.g., producing, isolating, purifying, synthesizing, and recombinantly manufacturing) polypeptides are well known to one of ordinary skill in the art.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The present composition encompasses amino acid substitutions in proteins and peptides, which do not generally alter the activity of the proteins or peptides (H. Neurath, R. L. Hill, The Proteins, Academic Press, New York, 1979). In one embodiment, these substitutions are “conservative” amino acid substitutions. The most commonly occurring substitutions are Ala/Ser, Val/Ile, Asp/Glu, Thr/Ser, Ala/Gly, Ala/Thr, Ser/Asn, Ala/Val, Ser/Gly, Ala/Pro, Lys/Arg, Asp/Asn, Leu/Ile, Leu/Val, Ala/Glu and Asp/Gly, in both directions

As to “conservatively modified variants” of amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Analogue as used herein denotes a peptide, polypeptide, or protein sequence which differs from a reference peptide, polypeptide, or protein sequence. Such differences may be the addition, deletion, or substitution of amino acids, phosphorylation, sulfation, acrylation, glycosylation, methylation, farnesylation, acetylation, amidation, and the like, the use of non-natural amino acid structures, or other such modifications as known in the art.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to the full length of the reference sequence, usually about 25 to 100, or 50 to about 150, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

As used herein, the term “prevention” means all of the actions by which the occurrence of the disease is restrained or retarded.

As used herein, the term “treatment” means all of the actions by which the symptoms of the disease have been alleviated, improved or ameliorated. In the present specification, “treatment” means that the symptoms of multiple myeloma are alleviated, improved or ameliorated by administration of the EPO proteins disclosed herein.

As used herein, the term “administration” means introduction of an amount of a predetermined substance into a patient by a certain suitable method. The composition disclosed herein may be administered via any of the common routes, as long as it is able to reach a desired tissue, for example, but is not limited to, intraperitoneal, intravenous, intramuscular, subcutaneous, intradermal, oral, topical, intranasal, intrapulmonary, or intrarectal administration. However, since peptides are digested upon oral administration, active ingredients of a composition for oral administration should be coated or formulated for protection against degradation in the stomach.

In the present specification, the term “subject” is those suspected of having multiple myeloma. However, any subject to be treated with the EPO proteins or the pharmaceutical composition disclosed herein is included without limitation. The subject is being treated with EPO to inhibit the primary cancer and not as a treatment for anemia related to the disease state or to administration of chemotherapy.

### EXAMPLES

The following non-limiting examples are provided for illustrative purposes only in order to facilitate a more complete understanding of representative embodiments now contemplated. These examples should not be construed to limit any of the embodiments described in the present specification, including those pertaining to the compounds, pharmaceutical compositions, or methods and uses disclosed herein.

Materials and Methods: ErepoXen™ (and a de-sialylated derivative) will be obtained from Xenetic Bioscience Inc. rhuEPO will be obtained from R&D Systems. Primary myeloma cell isolates will be obtained from the National Biobank on Multiple Myeloma, Norway. EPO toxicity towards primary myeloma cells will be estimated by semi-automated fluorescence microscopy based on the ScanR platform (Olympus (Misund et al., 2013, J Biomol Screen). EPOR expression on primary myeloma cells and cell lines will be estimated by flow-cytometry (Våtsveen et al., 2016). EPO activity towards myeloma cell lines will be estimated applying CellTiter Glo assays (Promega Inc.).

Example 1: EPO activity towards cell lines: Dilutions of rhuEPO—as well as of ErepoXen™ and its de-sialyalated derivative will be tested in 4 myeloma cell lines applying the CelltiterGlo assay. Two of the cell lines (e.g., INA-6 and ANBL-6) express high amounts of sell surface EPOR, whereas two cell lines express little or no cell surface EPOR (e.g., JJN3 and RPMI-8226).

The cell lines expressing high amounts of surface EPOR are expected to respond to both the ErepoXen™ and its de-sialyalated derivative as well as rhuEPO, by entering apoptosis. The cell lines expressing little to no surface EPOR are expected to be non-responsive to both the ErepoXen™ and its de-sialyalated derivative as well as rhuEPO.

Example 2: EPO activity towards primary myeloma cell isolates: Dilutions rhuEPO as well as of ErepoXen™ and its de-sialylated derivative will be tested in 20 primary myeloma isolates (CD138⁺ plasma cells) in the presence of human bone marrow stromal cells. The primary endpoints of analysis are toxicity of drugs towards myeloma cells (CD138⁺ cells) and towards bone marrow stromal cells.

ErepoXen™ and its de-sialylated derivative will show similar toxicity levels to the cell lines, however, those cells lines expressing high levels of EPOR are expected to respond to treatment by ErepoXen™ and its de-sialylated derivative, i.e., enter into apoptosis.

Example 3: Expression of EPOR on surface of primary myeloma (CD138⁺ cells) by flow cytometry: The same samples as tested for EPO sensitivity above will be analyzed for expression levels of cell surface EPOR by multi-parameter flow cytometry.

Cells that are sensitive to ErepoXen™ and its de-sialylated derivative will express high levels of cell surface EPOR in contrast to cells that lack sensitivity to ErepoXen™ and its de-sialylated derivative.

Example 4: Administration of ErepoXen™ to a test subject: ErepoXen™ is administered to a 38 year old male with multiple myeloma. The patient is selected for the study based on immunoassay biopsy testing of his cancer that determines that he has high levels of EPOR expression in the multiple myeloma cells. The patient is dosed with a lower level of ErepoXen™ than the standard dose of EPO given to treat anemia in cancer patients due to the slower clearance of ErepoXen™. The ErepoXen™ causes a reduction in tumor load as it acts directly on the primary cancer with high levels of EPOR expression. Adverse side effects are not observed.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub-combinations as are within their true spirit and scope.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. For instance, as mass spectrometry instruments can vary slightly in determining the mass of a given analyte, the term “about” in the context of the mass of an ion or the mass/charge ratio of an ion refers to +/−0.50 atomic mass unit. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only, and is not intended to limit the scope of the present invention, which is defined solely by the claims. Accordingly, the present invention is not limited to that precisely as shown and described.

1. A method of treating multiple myeloma in a subject in need thereof, the method comprising administering to the subject a pharmaceutical composition comprising an effective amount of an EPO conjugated with a water soluble polymer, wherein a myeloma sample from the subject has erythropoietin receptor expressed on the cell surface and wherein the EPOR receptor expression is higher than a predetermined threshold level.

2. The method of claim 1, wherein the erythropoietin receptor expression in the myeloma sample is measured using an immunoassay.

3. The method of claim 2, wherein the predetermined threshold level by ELISA is at least or above 0.5 pg/mg of sample protein, 1.5 pg/mg of sample protein, 5 pg/mg of sample protein, 50 pg/mg of sample protein or higher.

4. The method of claim 2, wherein the predetermined threshold level is a EpoR receptor density per cell and is at least 100 EpoR receptor copies per cell.

5. The method of claim 2, wherein the predetermined threshold level is a percent of EpoR positive myeloma cells (ICH) in the sample at least over 5%, 10% to 25% or greater than 25%.

6. The method of claim 1, wherein the erythropoietin receptor gene product expression in the myeloma sample is measured using PCR.

7. The composition of claim 1, wherein the EPO is conjugated to the water soluble polymer via a linking group.

8. The composition of claim 1, wherein the water soluble polymer is PEG, poly(2-ethyl 2-oxazoline), poly[oligo(ethylene glycol) methyl methacrylate], polyoxazoline, poly(N-(2-hydroxypropyl) methacrylamide, polyglycerol, poly(N-vinylpyrrolidone), polycarbonate, poly(carboxybetaine methacrylate), poly(sulfobetaine methacrylate) or poly(2-methyacryloyloxyethyl phosphorylcholine).

9. The method of claim 1, wherein the EPO is linked via an amine group at the N-terminus to a polysaccharide.

10. The method of claim 9, wherein the polysaccharide is selected from the group consisting of polysialic acid, heparin, hyaluronic acid, dextran, dextrin, hydroxyethyl starch, and chondroitin sulphate.

11. The method of claim 10, wherein the polysaccharide is polysialic acid.

12. The method of claim 10, wherein the polysialic acid is attached to the N-terminus of EPO at the reducing terminal unit of the polysialic acid.

13. The method of claim 1, wherein the EPO has at least 95% sequence identity to an amino acid sequence comprising SEQ ID NO:1.

14. The method of claim 13, wherein the EPO has an amino acid sequence comprising SEQ ID NO: 1.

15. The method of claim 13, wherein the EPO has an amino acid sequence comprising residues 28-193 of SEQ ID NO: 1.

16. The method of claim 1, wherein the subject has not received EPO treatment for anemia.

17. The method of claim 1, wherein the patient is suffering from concurrent anemia.

18. The method of claim 1, wherein the EPO conjugate has a systemic clearance at least 50% or lower as compared to EPO that is not conjugated with a water soluble polymer

19. A companion therapeutic-diagnostic kit comprising an antibody or EPOR binding partner for detecting erythropoietin receptor expression in a myeloma sample and a pharmaceutical composition comprising EPO conjugated to a polysialic acid.

20. A companion therapeutic-diagnostic kit comprising nucleic acid primers for detecting erythropoietin receptor gene product expression in a myeloma sample and a pharmaceutical composition comprising EPO conjugated to a polysialic acid.


SEQUENCE LISTING (1 sequence)

SEQ ID NO: 1 - protein, 193 aa, human
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR